(19) United States
(12) Patent Application Publication
(10) Pub. No.: US 2025/0342356 A1
(43) Pub. Date: Nov. 6, 2025

(54) DEEP LEARNING-BASED METHOD AND SYSTEM FOR PREDICTING FIRING PROPERTIES OF ANISOTROPIC MATERIAL BY USING INDENTATION RESPONSE DATA

(71) Applicant: SEOUL NATIONAL UNIVERSITY R&DB FOUNDATION, Seoul (KR)

(72) Inventors: Heung Nam HAN, Seoul (KR); Kyeong Jae JEONG, Seoul (KR)

(21) Appl. No.: 18/880,476

(22) PCT Filed: May 24, 2023
(86) PCT No.: PCT/KR2023/007074

§ 371 Date: Dec. 31, 2024

(30) Foreign Application Priority Data
Jul. 29, 2022 (KR) 10-2022-0094862
May 8, 2023 (KR) 10-2023-0059225

(51) Int. Cl. G06N 3/08

(52) U.S. Cl. CPC G06N 3/08

(57) ABSTRACT

Provided is a deep learning-based method and system for predicting the plastic properties of an anisotropic material by using indentation response data, which is capable of easily and quickly obtaining the plastic properties of an anisotropic material in a non-destructive manner. The method includes preparing a plurality of data sets, which are composed of indentation response data for learning and plastic properties data for learning, about an anisotropic material for learning; performing deep learning on a computer system by using the indentation response data for learning as input values and using the plastic properties data for learning as output values; providing actual indentation response data about a to-be-predicted anisotropic material; and inputting the actual indentation response data into the deep-learned computer system to predict the plastic properties of the to-be-predicted anisotropic material.

### TECHNICAL FIELD

The technical idea of the present invention relates to a method of predicting the plastic properties of an anisotropic material, and more particularly to a deep learning-based method and system for predicting the plastic properties of an anisotropic material by using indentation response data.

### BACKGROUND ART

When a metal material is plastically processed by rolling, drawing, extrusion, etc., or formed into a fiber-reinforced body, or a film layer is deposited or coated thereon, a texture is formed and grown, which may result in plastic anisotropy. The plastic anisotropy can change formability required in forming processes such as bending, tension, and deep drawing, so it is very important to accurately measure or predict the plastic anisotropy.

Conventionally, to measure the plastic anisotropy of a material, a uniaxial tensile test or a uniaxial compression test was performed several times while changing the angle. However, these tests are expensive and time-consuming, and are performed essentially while destroying specimens, so they have limitations in being applied to specimens with limited volume or small quantities. Therefore, there is a need for a method to analyze plastic anisotropy more easily and quickly in a non-destructive manner.

### DISCLOSURE

### Technical Problem

The present invention has been made in view of the above problems, and it is one object of the present invention to provide a deep learning-based method and system for predicting the plastic properties of an anisotropic material by using indentation response data, the method and system capable of easily and quickly obtaining the plastic properties of an anisotropic material in a non-destructive manner.

It will be understood that the technical problems are only provided as examples, and the technical idea of the present invention is not limited thereto.

### Technical Solution

In accordance with an aspect of the present invention, the above and other objects can be accomplished by the provision of a deep learning-based method and system for predicting the plastic properties of an anisotropic material by using indentation response data, the method and system capable of easily and quickly obtaining the plastic properties of an anisotropic material in a non-destructive manner.

In accordance with an embodiment of the present invention, the deep learning-based method of predicting the plastic properties of an anisotropic material using indentation response data may include: preparing a plurality of data sets, which are composed of indentation response data for learning and plastic properties data for learning, about an anisotropic material for learning; performing deep learning on a computer system by using the indentation response data for learning as input values and using the plastic properties data for learning as output values; providing actual indentation response data about a to-be-predicted anisotropic material; and predicting plastic properties of the to-be-predicted anisotropic material by inputting the actual indentation response data into the deep-learned computer system.

In accordance with an embodiment of the present invention, the deep learning-based method of predicting the plastic properties of an anisotropic material using indentation response data may include: providing a computer system deep-learned using the indentation response data for learning as input values and the plastic properties data for learning as output values, in the plural data sets, which are composed of indentation response data for learning and plastic properties data for learning, for the anisotropic material for learning; providing actual indentation response data about a to-be-predicted anisotropic material; and inputting the actual indentation response data into the deep-learned computer system to predict the plastic properties of the to-be-predicted anisotropic material.

In accordance with an embodiment of the present invention, the deep learning-based system for predicting the plastic properties of an anisotropic material using indentation response data may include a finite element simulation performance module and a deep learning performance module, and may include a) a step of preparing a plurality of data sets, which are composed of indentation response data for learning and plastic properties data for learning, about an anisotropic material for learning; b) a step of performing deep learning on a computer system by using the indentation response data for learning as input values and using the plastic properties data for learning as output values; c) a step of providing actual indentation response data about a to-be-predicted anisotropic material; and d) a step of inputting the actual indentation response data into the deep-learned computer system to predict the plastic properties of the to-be-predicted anisotropic material. Here, step a) may be performed by the finite element simulation performance module, and steps b) and d) may be performed by the deep learning performance module.

### Advantageous Effects

A deep learning-based method of predicting the plastic properties of an anisotropic material by using indentation response data according to the technical idea of the present invention uses a non-destructive and highly efficient indentation test instead of a tensile test involving the destruction of a material, and uses an artificial neural network system capable of correlating an indentation test results with the plastic properties, thereby easily and quickly obtaining the plastic properties of an anisotropic material in a non-destructive manner.

It will be understood that the effects of the present invention are only provided as examples, and the scope of the present disclosure is not limited thereto.

### DESCRIPTION OF DRAWINGS

FIG. 1 is a flowchart illustrating a deep learning-based method of predicting the plastic properties of an anisotropic material by using indentation response data according to an embodiment of the present invention.

FIG. 2 is a schematic diagram illustrating a deep learning-based system for predicting the plastic properties of an anisotropic material using indentation response data according to an embodiment of the present invention.

FIG. 3 is a flowchart illustrating a step of preparing a plurality of data sets in the deep learning-based method of predicting the plastic properties of an anisotropic material using indentation response data, as shown in FIG. 1, according to an embodiment of the present invention.

FIGS. 4 and 5 are graphs showing displacement due to the indentation calculated using the finite element simulation, in the deep learning-based method of predicting the plastic properties of an anisotropic material by using indentation response data according to an embodiment of the present invention.

FIGS. 6 to 8 illustrate the linearity between displacements due to indentation obtained using a finite element simulation, in the deep learning-based method of predicting the plastic properties of an anisotropic material by using indentation response data according to an embodiment of the present invention.

FIGS. 9 to 11 illustrate graphs comparing finite element simulation results and actual simulation results, in the deep learning-based method of predicting the plastic properties of an anisotropic material by using indentation response data according to an embodiment of the present invention.

FIG. 12 illustrates a schematic diagram of an artificial neural network that performs deep learning, in the deep learning-based method of predicting the plastic properties of an anisotropic material by using indentation response data according to an embodiment of the present invention.

FIG. 13 illustrates the effect of data linearity on the performance of an artificial neural network performing deep learning, in the deep learning-based method of predicting the plastic properties of an anisotropic material by using indentation response data according to an embodiment of the present invention.

FIGS. 14 to 16 illustrate graphs comparing plasticity characteristic prediction results, obtained using an artificial neural network that is deep-learned by the deep learning-based method of predicting the plastic properties of an anisotropic material by using indentation response data according to an embodiment of the present invention, and experimental results.

FIG. 17 illustrates plastic properties predicted using an artificial neural network deep-learned by the deep learning-based method of predicting the plastic properties of an anisotropic material by using indentation response data according to an embodiment of the present invention.

### BEST MODE

Hereinafter, preferred embodiments of the present invention will be described in detail with reference to the accompanying drawings. Embodiments of the present disclosure are provided to more completely explain the technical idea of the present disclosure to those skilled in the art, and the following embodiments may be modified in many different forms, but the scope of the technical idea of the present disclosure is not limited to the following embodiments. Rather, the embodiments are provided to make the disclosure thorough and complete and to fully convey the technical idea of the disclosure to those skilled in the art. Like reference numerals in the specification denote like elements. Further, various elements and regions in the drawings are schematically drawn. Therefore, the technical idea of the invention is not limited by the relative size or spacing drawn in the accompanying drawings.

The indentation technique measures hardness using the size and depth of indentation formed by applying pressure to a specimen with an indenter, and has the advantage of being able to precisely measure the plastic properties of a small specimen. The instrumented indentation test (IIT) is a test method that continuously records the load applied by an indenter and the indentation depth of the indenter, and can measure the hardness, elastic modulus, and other hardening characteristics of a target material through a load-depth curve and its analysis method. In addition, a protocol for analyzing a global uniaxial tensile behavior using the finite element method based on local load-depth curves obtained from high-resolution nano-indentation test data has been proposed. The plastic properties of the target material can be inversely estimated based on finite element simulation and optimization algorithms from the load-depth curves obtained by instrumented indentation tests using a spherical or sharp indenter. In addition, to solve the non-uniqueness caused when determining the mechanical properties numerically from the load-depth curve, additional indentation information such as dual indentation is used, or vertical residual indentation marks, such as pile-up or sink-in, around the indentation are considered. In addition, instead of the load-depth curve, the pile-up/sink-in, in-plane displacement, and residual indentation trace profiles are used to obtain the plastic properties.

However, the prediction of plastic properties based on indentation data has been studied extensively for isotropic materials, but research on anisotropic materials close to real materials is still insufficient. As a conventional method, there is a method of obtaining the plastic properties of anisotropic materials from indentation data under the assumption of transverse isotropy so as to simplify unknown material parameters, but this method has a limitation in that it is difficult to predict the overall mechanical anisotropy. In addition, conventionally, load-depth curves and residual vertical displacement were considered, but residual in-plane displacement field was not considered, which has limitations. Therefore, it can be proposed to use an artificial neural network (NN), which shows excellent performance, as a universal approximator to extract general anisotropic plasticity from various indentation results. The artificial neural network can model the complex relationship between input and output values with very high accuracy based on statistical deep learning algorithms without using equations. To date, research using artificial neural networks for anisotropic materials has been insufficient.

The present inventors have established a general framework for analyzing the plastic properties of bulk materials. By using this framework, the anisotropic properties can be comprehensively analyzed using finite element-deep learning modeling from the spherical indentation response consisting of the load-depth curve, pile-up/sink-in, and in-plane displacement fields. In the finite element-deep learning modeling, the anisotropic plastic properties acquired by deep learning that used an artificial neural network with hyperparameters adjusted were compared with actual experimental results, and the results showed that the finite element-deep learning modeling was robust and effective.

Hereinafter, the plastic properties of an anisotropic material are described in detail.

The elastic behavior of a continuous material such as a metal is as shown in Mathematical Equation 1 known as Hooke's law:

$$σ = E ε \qquad [ Mathematical Equation 1 ]$$

In Mathematical Equation 1, σ is a stress, E is an elastic modulus, and ε is a strain rate. To simplify and focus on the analysis of the plastic properties of a material, the influence on the indentation curve may be ignored, and the Poisson's ratio may be set to 0.3.

To describe the strain hardening behavior from the starting point of plastic yielding, a nonlinear isotropic hardening model may be adopted, and the Swift equation of Mathematical Equation 2, which is a power law, may be introduced in the user-defined subroutine UHARD:

$${\overline{σ}}_{Swift} ( \overline{{ε}_{p}} ) = {k ( {ε}_{0} + \overline{{ε}_{p}} )}^{n} \qquad [ Mathematical Equation 2 ]$$

In Mathematical Equation 2, σ̅_{Swift} is a Swift effective stress, ε̅ₚ is an equivalent plastic strain, k is a strength coefficient, ε₀ is a strain parameter, and n is a strain hardening exponent. Hereinafter, k, ε₀, and n are referred to as Swift hardening parameters.

In the present invention, the sixth-order polynomial yield criterion may be applied as a constitutive equation. Hereinafter, the sixth-order polynomial yield criterion is referred to as Poly6. The 3D shape expression for the yield criterion of the Poly6 model is as Mathematical Equation 3:

$$f = {a}_{1} {\sum }_{xx}^{6} + {α}_{2} {\sum }_{xx}^{5} {\sum }_{yy} + {α}_{3} {\sum }_{xx}^{4} {\sum }_{yy}^{2} + {α}_{4} {\sum }_{xx}^{3} {\sum }_{yy}^{3} + {α}_{5} {\sum }_{xx}^{2} {\sum }_{yy}^{4} + {α}_{6} {\sum }_{xx} {\sum }_{yy}^{5} + {α}_{7} {\sum }_{yy}^{6} + ( {α}_{8} {\sum }_{xx}^{4} + {α}_{9} {\sum }_{xx}^{3} {\sum }_{yy} + {α}_{10} {\sum }_{xx}^{2} {\sum }_{yy}^{2} + {α}_{11} {\sum }_{xx} {\sum }_{yy}^{3} + {α}_{12} {\sum }_{yy}^{2} ) {\sum }_{xy}^{2} + ( {α}_{13} {\sum }_{xx}^{2} + {α}_{14} {\sum }_{xx} {\sum }_{yy} + {α}_{15} {\sum }_{yy}^{2} ) {( {\sum }_{xy}^{2} )}^{2} + {{α}_{16} ( {σ}_{xy}^{2} + {σ}_{yz}^{2} + {σ}_{zx}^{2} )}^{3} = {σ}_{0}^{6} \qquad [ Mathematical Equation 3 ]$$

$$\{ \begin{matrix}{\sum }_{xx} = {σ}_{xx} - {σ}_{zz} \\ {\sum }_{yy} = {σ}_{yy} - {σ}_{zz} \\ {\sum }_{xy}^{2} = {σ}_{xy}^{2} + {σ}_{yz}^{2} + {σ}_{zx}^{2}\end{matrix}$$

In Mathematical Equation 3, σₓₓ, σ_{yy} and σ_{zz} are vertical stresses (normal stresses) related to the vertical direction, and σ_{xy}, σ_{yz} and σ_{zx} are shear stresses. a₁ to a₁₆ are independent Poly6 anisotropy parameters, and may be defined based on uniaxial tensile test data and biaxial tensile test data.

The most common data set considered in this method is as shown in Mathematical Equation 4:

$$Biaxial curve set : \{ {σ}_{0} , {r}_{0} , {σ}_{b} , {r}_{b} , {σ}_{90} , {r}_{90} \} \qquad [ Mathematical Equation 4 ]$$

$$Directional data set : \{ {σ}_{15} , {r}_{15} , {σ}_{30} , {r}_{30} , {σ}_{45} , {r}_{45} , {σ}_{60} , {r}_{60} , {σ}_{75} , {r}_{75} \}$$

In Mathematical Equation 4, σ_{b} is a balanced biaxial yield stress, and r_{b} is the r-value defined as r_{b}=dε_{yy}/dεₓₓ.

Mathematical Equation 5 shows a uniaxial stress state function dependent upon an angle.

$$σ ( θ ) = {σ}_{θ} ( {cos}^{2} θ , {sin}^{2} θ , sin θ cos θ ) \qquad [ Mathematical Equation 5 ]$$

In Mathematical Equation 5, θ refers to an angle in a clockwise or counterclockwise direction from a reference direction, for example, a rolling direction (RD).

The r-value of a specimen is a ratio of a transverse strain (a strain component perpendicular to the direction of an indentation load) to a thickness strain. Mathematical Equation 6 shows an r-value state function dependent upon an angle.

$${r}_{θ} = \frac{{sin}^{2} θ d {ϵ}_{xx} + {cos}^{2} θ d {ϵ}_{yy} - 2 sin θ cos θ d {ϵ}_{xy}}{- ( d {ϵ}_{xx} + d {ϵ}_{yy} )} \qquad [ Mathematical Equation 6 ]$$

Mathematical Equation 7 shows an r-value considering the normality rule, the rigid-plastic approximation, and the volume invariance of the plastic strain.

$$( {r}_{θ} + {sin}^{2} θ ) \frac{\partial f}{\partial {σ}_{xx}} ( σ ( θ ) ) + ( {r}_{θ} + {cos}^{2} θ ) \frac{\partial f}{\partial {σ}_{yy}} ( σ ( θ ) ) - sin θ cos θ \frac{\partial f}{\partial {σ}_{xy}} ( σ ( θ ) ) = 0 \qquad [ Mathematical Equation 7 ]$$

Hereinafter, a deep learning-based method and system for predicting the plastic properties of an anisotropic material by using indentation response data by using indentation response data according to the technical idea of the present invention is described.

According to the technical idea of the present invention, a method and system for predicting the plastic properties of an anisotropic material based on deep learning using indentation response data which can easily and quickly acquire the plastic properties of the anisotropic material in a non-destructive manner are provided.

The deep learning-based method of predicting the plastic properties of an anisotropic material using indentation response data may include a step of preparing a plurality of data sets, which are composed of indentation response data for learning and plastic properties data for learning, about an anisotropic material for learning; a step of performing deep learning on a computer system by using the indentation response data for learning as input values and using the plastic properties data for learning as output values; a step of providing actual indentation response data about a to-be-predicted anisotropic material; and a step of inputting the actual indentation response data into the deep-learned computer system to predict the plastic properties of the to-be-predicted anisotropic material.

The step of preparing the plural data sets may include a step of providing the tensile properties of the anisotropic material for learning; a step of obtaining a poly6 anisotropy parameter, elastic modulus, and isotropic hardening parameter from the tensile properties of the anisotropic material for learning; a step of performing a finite element simulation using the poly6 anisotropy parameter, the elastic modulus, and the isotropic hardening parameter; and a step of obtaining the indentation response data for learning about the anisotropic material for learning as a result of performing the finite element simulation.

The tensile properties may include the tensile stress and r-value of the anisotropic material for learning.

The poly6 anisotropy parameter may be obtained from the following equation:

$$f = {a}_{1} {\sum }_{xx}^{6} + {α}_{2} {\sum }_{xx}^{5} {\sum }_{yy} + {α}_{3} {\sum }_{xx}^{4} {\sum }_{yy}^{2} + {α}_{4} {\sum }_{xx}^{3} {\sum }_{yy}^{3} + {α}_{5} {\sum }_{xx}^{2} {\sum }_{yy}^{4} + {α}_{6} {\sum }_{xx} {\sum }_{yy}^{5} + {α}_{7} {\sum }_{yy}^{6} + ( {α}_{8} {\sum }_{xx}^{4} + {α}_{9} {\sum }_{xx}^{3} {\sum }_{yy} + {α}_{10} {\sum }_{xx}^{2} {\sum }_{yy}^{2} + {α}_{11} {\sum }_{xx} {\sum }_{yy}^{3} + {α}_{12} {\sum }_{yy}^{2} ) {\sum }_{xy}^{2} + ( {α}_{13} {\sum }_{xx}^{2} + {α}_{14} {\sum }_{xx} {\sum }_{yy} + {α}_{15} {\sum }_{yy}^{2} ) {( {\sum }_{xy}^{2} )}^{2} + {{α}_{16} ( {σ}_{xy}^{2} + {σ}_{yz}^{2} + {σ}_{zx}^{2} )}^{3} = {σ}_{0}^{6}$$

$$\{ \begin{matrix}{\sum }_{xx} = {σ}_{xx} - {σ}_{zz} \\ {\sum }_{yy} = {σ}_{yy} - {σ}_{zz} \\ {\sum }_{xy}^{2} = {σ}_{xy}^{2} + {σ}_{yz}^{2} + {σ}_{zx}^{2}\end{matrix}$$

where σₓₓ, σ_{yy} and σ_{zz} are vertical stresses related to the vertical direction, and σ_{xy}, σ_{yz} and σ_{zx} are shear stresses.

The elastic modulus may include the Young's modulus (E) and Poisson's ratio (v) of the anisotropic material for learning.

The isotropic hardening parameter may include a strength coefficient (k), strain parameter (ε₀), and strain hardening exponent (n) obtained from the following equation:

$${\overline{σ}}_{Swift} ( \overline{{ε}_{p}} ) = {k ( {ε}_{0} + \overline{{ε}_{p}} )}^{n}$$

where σ̅_{Swift} is a Swift effective stress, and ε̅ₚ is an equivalent plastic strain.

After the step of obtaining the poly6 anisotropy parameter, a step of evaluating whether the poly6 anisotropy parameter of the anisotropic material for learning satisfies a convexity for the yield criterion of the anisotropic material for learning may be further included.

The step of performing the finite element simulation may be performed for a spherical indenter.

The output values of the finite element simulation may include a load-depth curve, in-plane displacement field information, and vertical displacement field information from the results of the anisotropic material for learning.

The indentation response data for learning may include indentation load data, radial displacement data, and vertical displacement data for an indentation formed by indenting the anisotropic material for learning.

The indentation load data may include a load value for the depth of the indentation.

The radial displacement data may include a radial displacement value for an angle from a reference direction of the indentation.

The radial displacement data may include a radial displacement value at a separation distance that is a multiple of the radius (R) of the indenter from the center of the indentation.

The vertical displacement data may include a vertical displacement for the angle from the reference direction of the indentation.

The vertical displacement data may include a vertical displacement value at a distance of a radius (R) of the indenter from the center of the indentation.

The actual indentation response data may include indentation load data, radial displacement data, and vertical displacement data for indentation formed by indenting the to-be-predicted anisotropic material.

The plastic properties of the to-be-predicted anisotropic material may include the poly6 anisotropy parameter, elastic modulus, and isotropic hardening parameter of the to-be-predicted anisotropic material.

After the step of predicting the step of predicting the plastic properties of the to-be-predicted anisotropic material, a step of evaluating whether the poly6 anisotropy parameter of the to-be-predicted anisotropic material satisfies the convexity for the yield criterion of the to-be-predicted anisotropic material may be further included.

After the step of predicting the plastic properties of the to-be-predicted anisotropic material, a step of comparing the predicted plastic properties of the to-be-predicted anisotropic material with the actual plastic properties of the to-be-predicted anisotropic material may be further included.

At least one of the anisotropic material for learning and the to-be-predicted anisotropic material may include a metal alloy.

The deep learning-based method of predicting the plastic properties of an anisotropic material using indentation response data may include a step of providing a computer system that performs deep learning using the indentation response data for learning as input values and the plastic properties data for learning as output values, in a plurality of data sets, which are composed of indentation response data for learning and plastic properties data for learning, about the anisotropic material for learning; a step of providing actual indentation response data about a to-be-predicted anisotropic material; and a step of inputting the actual indentation response data into the deep-learned computer system to predict the plastic properties of the to-be-predicted anisotropic material. This case is also applicable when there is a time gap between the time at which the computer system performs deep learning and the time at which the plastic properties are predicted.

The deep learning-based system for predicting the plastic properties of an anisotropic material using indentation response data includes a finite element simulation performance module and a deep learning performance module, and includes a) a step of preparing a plurality of data sets, which are composed of indentation response data for learning and plastic properties data for learning, about an anisotropic material for learning; b) a step of performing deep learning on a computer system by using the indentation response data for learning as input values and using the plastic properties data for learning as output values; c) a step of providing actual indentation response data about a to-be-predicted anisotropic material; and d) a step of inputting the actual indentation response data into the deep-learned computer system to predict the plastic properties of the to-be-predicted anisotropic material. Here, step a) may be performed by the finite element simulation performance module, and steps b) and d) may be performed by the deep learning performance module.

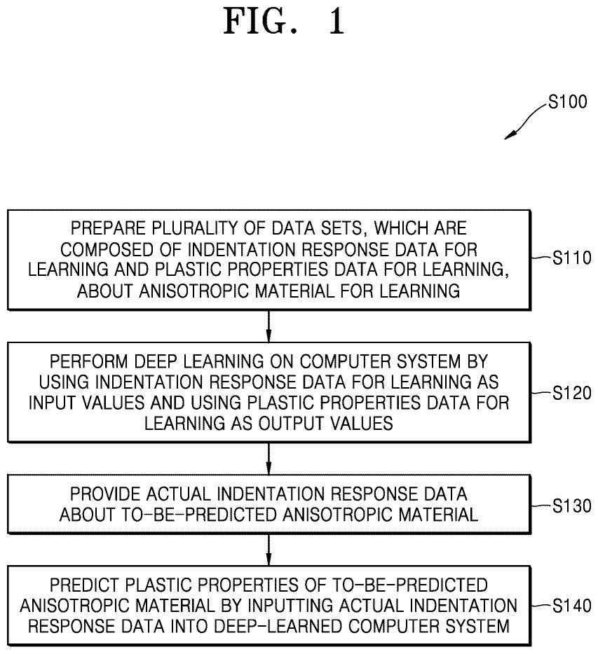

FIG. 1 is a flowchart illustrating a deep learning-based method of predicting the plastic properties of an anisotropic material by using indentation response data according to an embodiment of the present invention.

Referring to FIG. 1, a deep learning-based method (S100) of predicting the plastic properties of an anisotropic material includes a step (S110) of preparing a plurality of data sets, which are composed of indentation response data for learning and plastic properties data for learning, about an anisotropic material for learning; a step (S120) of performing deep learning on a computer system by using the indentation response data for learning as input values and using the plastic properties data for learning as output values; a step (S130) of providing actual indentation response data about a to-be-predicted anisotropic material; and a step (S140) of inputting the actual indentation response data into the deep-learned computer system to predict the plastic properties of the to-be-predicted anisotropic material.

At least one of the anisotropic material for learning and the to-be-predicted anisotropic material may include a material having plastic anisotropy, for example, a metal alloy, for example, an ultra-high strength steel or an aluminum alloy, for example at least one of DP780, TBF1050, AA7075 and AA 2090. However, these are provided only examples, and the technical idea of the present invention is not limited thereto.

In this specification, when indentation response data and plastic properties data are included in data sets for deep learning, the term “for learning” will be added to the name, and when they are measured by actual experiments, the term “actual” will be added to the name. Of course, actual indentation response data and actual plastic property data can be used in the deep learning of artificial neural networks to improve learning accuracy.

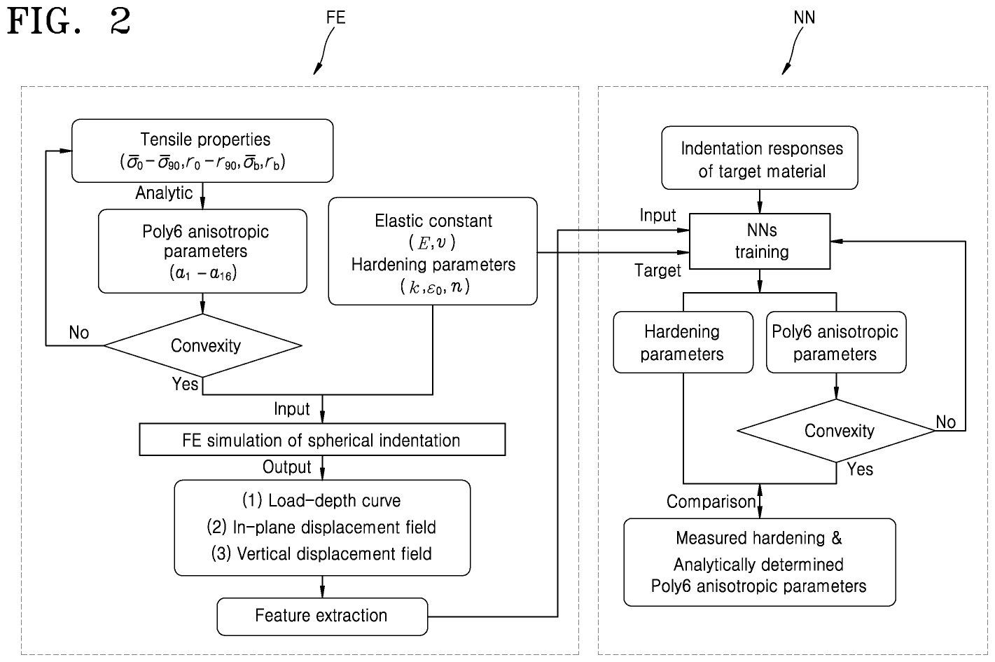

FIG. 2 is a schematic diagram illustrating a deep learning-based system for predicting the plastic properties of an anisotropic material using indentation response data according to an embodiment of the present invention.

Referring to FIG. 2, the deep learning-based system for predicting the plastic properties of an anisotropic material may include a finite element simulation performance module (FE) and a deep learning performance module (NN).

The finite element simulation performance module (FE) may perform a finite element simulation to derive indentation response elements of an anisotropic material for learning. The finite element simulation performance module (FE) may verify whether an indentation response simulation result is consistent with an experimental indentation result, thereby verifying the validity and reliability of data collection for deep learning of an artificial neural network. The finite element simulation performance module (FE) that performs the finite element simulation may be configured as a computer system.

The finite element simulation performance module (FE) may perform the step (S110) of preparing a plurality of data sets as shown in FIG. 1.

The indentation response data for learning and the plastic properties data for learning included in the data sets may be, for example, 100 sets or more, in terms of securing the accuracy of the output values, may be, for example, 1,000 sets or more to further increase the accuracy, or may be, for example 50,000 sets or more. However, the technical idea of the present invention is not limited thereto. As the number of the data sets increases, the accuracy of output values increases, but it can be appropriately selected considering time and cost. The data sets may be provided through actual experimental results, or may be provided by repeatedly performing finite element simulation while changing input values.

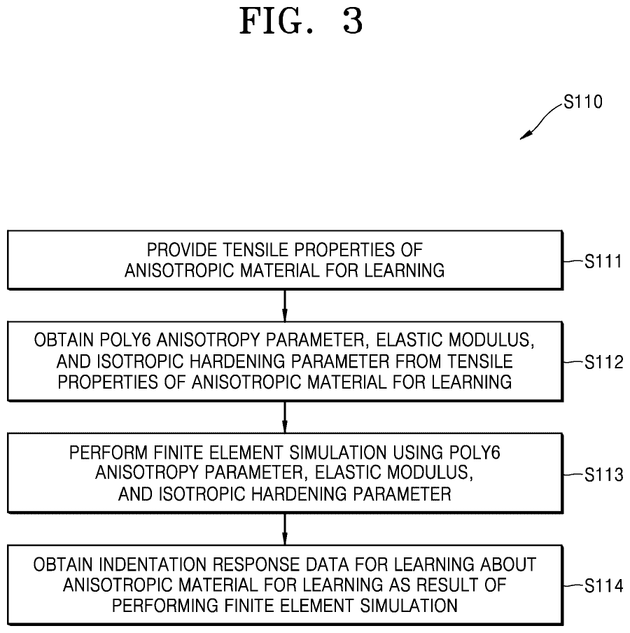

FIG. 3 is a flowchart illustrating the step of preparing a plurality of data sets in the deep learning-based method of predicting the plastic properties of an anisotropic material using indentation response data, as shown in FIG. 1, according to an embodiment of the present invention.

Referring to FIG. 3, the step (S110) of preparing the plural data sets may include a step (S111) of providing the tensile properties of the anisotropic material for learning; a step (S112) of obtaining a poly6 anisotropy parameter, elastic modulus, and isotropic hardening parameter from the tensile properties of the anisotropic material for learning; a step (S113) of performing a finite element simulation using the poly6 anisotropy parameter, the elastic modulus, and the isotropic hardening parameter; and a step (S114) of obtaining the indentation response data for learning about the anisotropic material for learning as a result of performing the finite element simulation.

In the step (S111) of providing the tensile properties of the anisotropic material for learning, the tensile stress (σ₀˜σ₉₀, σ_{b}) and r-value (r₀˜r₉₀, r_{b}) of the anisotropic material for learning are provided as examples of the tensile properties of the anisotropic material for learning as shown in Mathematical Equation 4. The tensile stress and the r-value of the anisotropic material for learning may include test results obtained by directly conducting a tensile experiment on the anisotropic material for learning.

In the obtaining step (S112), the plastic properties data for learning is obtained from the tensile properties, and for example, the poly6 anisotropy parameter (a₁˜a₁₆), elastic modulus, and an isotropic hardening parameter may be obtained therefrom. The poly6 anisotropy parameter may be obtained from Mathematical Equation 3. From the tensile properties, the elastic modulus and isotropic hardening parameter of the anisotropic material for learning are obtained. The elastic modulus may include Young's modulus (E) and Poisson's ratio (v). The isotropic hardening parameter may include the strength coefficient (k), strain parameter (ε₀), and strain hardening exponent (n) obtained from Mathematical Equation 2.

After the step (S112) of obtaining a poly6 anisotropy parameter, a step of evaluating whether the poly6 anisotropy parameter of the anisotropic material for learning satisfies the convexity for the yield criterion of the anisotropic material for learning may be further included. When the poly6 anisotropy parameter does not satisfy the convexity, a step of providing tensile properties through feedback is performed again. When the poly6 anisotropy parameter satisfies the convexity, the poly6 anisotropy parameter, the elastic modulus, and the isotropic hardening parameter are set as input values for the finite element simulation.

In the step (S113) of performing the finite element simulation, the finite element simulation is performed using the poly6 anisotropy parameter, the elastic modulus, and the isotropic hardening parameter as input values. The step (S113) of performing the finite element simulation may be performed for a spherical indenter. However, this is exemplary and the technical idea of the present invention is not limited thereto. The output values of the finite element simulation may include a load-depth curve of the anisotropic material for learning, in-plane displacement field information of the surface, and vertical displacement field information.

In the step (S114) of obtaining the indentation response data for learning, a process of extracting feature data from the output values is performed for subsequent deep learning execution, and thus, indentation response data for learning may be acquired as the feature data.

The indentation response data for learning may include indentation load data, radial displacement data, and vertical displacement data of the anisotropic material for learning.

Referring to FIG. 2 again, the deep learning performance module (NN) may perform the step (S120) of performing deep learning as shown in FIG. 1.

The deep learning performance module (NN) may cause the computer system to perform deep learning using the indentation response data for learning as input values and the plastic properties data for learning as output values. The deep learning may be performed at least once, and, accordingly, the artificial neural network equipped in the computer system may be deep-learned.

The deep learning performance module (NN) that performs the deep learning may be configured as a computer system. The finite element simulation performance module (FE) and the deep learning performance module (NN) may be configured as the same computer system or separate computer systems. The computer system referred to in this specification includes an artificial intelligence program, an artificial neural network program or any system equipped with the program.

Referring to FIGS. 1 and 2 again, a step (S130) of providing actual indentation response data about a to-be-predicted anisotropic material is performed. The actual indentation response data may include indentation load data, radial displacement data, and vertical displacement data of the to-be-predicted anisotropic material. The actual indentation response data may include a test result obtained by directly indenting the to-be-predicted anisotropic material. For example, the indentation load data, the radial displacement data, and the vertical displacement data may be obtained by directly indenting the to-be-predicted anisotropic material to obtain a load-depth curve, in-plane displacement field information of a surface, and vertical displacement field information, and then extracting feature data therefrom. Alternatively, the actual indentation response data may be obtained by performing the finite element simulation.

Referring to FIGS. 1 and 2 again, a step (S140) of inputting the actual indentation response data into the deep-learned computer system to predict the plastic properties of the to-be-predicted anisotropic material is performed. The plastic properties may include the poly6 anisotropy parameter, elastic modulus, and isotropic hardening parameter of the to-be-predicted anisotropic material.

After the step (S140) of predicting the plastic properties of the to-be-predicted anisotropic material, a step of evaluating whether the poly6 anisotropy parameter of the to-be-predicted anisotropic material predicted by the deep-learned computer system satisfies the convexity for the yield criterion of the to-be-predicted anisotropic material may be further included. When the poly6 anisotropy parameter does not satisfy the convexity, deep learning is performed again through feedback. When the poly6 anisotropy parameter satisfies the convexity, the poly6 anisotropy parameter of the to-be-predicted anisotropic material, the elastic modulus, and the isotropic hardening parameter are outputted as the plastic properties of the to-be-predicted anisotropic material.

The poly6 anisotropy parameter may include parameters (a₁˜a₁₆) as shown in Mathematical Equation 3. The elastic modulus may include Young's modulus (E) and Poisson's ratio (v). The isotropic hardening parameter may include the strength coefficient (k), strain parameter (ε₀), and strain hardening exponent (n) as shown in Mathematical Equation 2.

The step of evaluating the convexity of the to-be-predicted anisotropic material may be performed also in the step (S120) of performing deep learning.

Optionally, after the step of predicting the plastic properties of the to-be-predicted anisotropic material, a step of comparing the predicted plastic properties of the to-be-predicted anisotropic material with the actual plastic properties of the to-be-predicted anisotropic material may be further included. The actual plastic properties may be test results obtained by directly experimenting the to-be-predicted anisotropic material. As a result of the comparison, when a difference between the predicted plastic properties and the actual plastic properties is within a certain error rate range, the deep learning may be terminated, and when it is outside the certain error rate range, the predicting step may be performed again.

The above-described deep learning is a term that is generally widely used in the field of artificial intelligence, and the present invention does not specifically limit its method. According to an embodiment of the present invention, by inputting the data sets into an artificial neural network (or artificial intelligence) and performing deep learning (or machine learning), an artificial neural network that predicts plastic properties data as output values when indentation response data is input as input values may be constructed. The principle by which this artificial neural network is constructed is similar to the regression problem for general linear functions. That is, it creates a function that informs the relationship between the input values and output values of the given data sets. The deep learning method of the artificial neural network is a widely known method, such as general deep learning, and the present invention may adopt any method among the already known methods.

As an example of the deep learning, the case where the number of nodes in a first column included in an input layer of the artificial neural network, the number of nodes in a second column included in a hidden layer is 45, the number of nodes in a third column is 45, the number of nodes in a fourth column is 45, and the number of nodes in a fifth column included in an output layer is 19 is explained.

Input values are entered in the first column, and then each node in the first column is connected to each node in the second column through each line, and the node values in the second column are determined using the input values in the first column through each line. A first node at the top of the second column follows the same calculation as Relation A:

$$value 1 = 1 / ( 1 + exp ( - ( a1\_ 1 X ε 1 + a1\_ 2 X ε 2 + … + a1\_ 19 X ε 19 ) ) ] \qquad [ Relation A ]$$

In Relation A, value1 represents a value of the first node of the second column, each of a1_1 to a1_19 represents the weight of each input value for determining the value of the first node of the second column, and ε1 to ε19 respectively represent 19 input values.

Next, the second node of the second column also follows the same calculation, and specifically follows the calculation of Relation B below:

$$value 2 = 1 / ( 1 + exp ( - ( a2\_ 1 X ε 1 + a2\_ 2 X ε 2 + + a2\_ 19 X ε 19 ) ) ] \qquad [ Relation B ]$$

In Relation B, value2 represents the value of the second node in the second column, each of a2_1 to a2_19 represents the weight of each input value for determining the values of the second nodes in the second column, and ε1 to ε19 respectively represent 19 input values.

This same process may be repeated from the third column to the last fifth column. Next, by changing values of unknowns indicated by a#_# (where # is a number), the function called artificial neural network finds the relationship between the input and output of the actual data.

For this, first, a random number is assigned to the a#_#, and a difference between the calculated output value and the output of the actual data is calculated, and the values of the a#_# are modified using a gradient descent method to reduce the difference. This process of modifying the values of unknowns using the difference between the output values is called back-propagation. The process of the artificial neural network finding relationships between actual data while performing this back-propagation corresponds to the deep learning of artificial intelligence.

Through the deep learning process of artificial intelligence as described above, a predicted value can be obtained immediately by simply inputting measured input values into the deep-learned computer system without separate work by the actual user according to the present invention, unlike a conventional method that requires separate work by an actual user. Therefore, since it can be easily used in combination with existing experimental equipment, a dramatic reduction in time and cost is possible.

According to the technical idea of the present invention, a deep artificial neural network (NN) model capable of identifying a nonlinear correlation between discontinuous input data and output data may be applied. To improve the performance of the artificial neural network model, the basic hyperparameters, i.e., the number of hidden layers and the number of neurons per hidden layer, may be tuned using the Bayesian optimization algorithm to optimize the artificial neural network model structure. The number of neurons per hidden layer may be set to be the same, and by optimizing the hyperparameters, a 3-layer model with 45 neurons for each of the hidden layers may be applied. The prepared data set may be randomly assigned to the deep learning, validation, and test sets. In the deep learning process, the mean square error (MSE) of the validation set may be monitored to determine the completion of deep learning while avoiding overfitting. The performance of the learned artificial neural network may be evaluated using the test data sets excluded during deep learning. New information not used in deep learning may be provided to a deep-learned artificial neural network, and the calculated and measured parameters may be compared. Since the initial weights, biases, and data sets partitions for deep learning may be configured differently, the deep learning may be repeated independently multiple times, for example, 5 times, and the mean square error and output may be calculated to ensure sufficient generalization. The maximum epoch of deep learning may be set to, for example, 30 or more, for example, 200, which is large enough to stabilize the artificial neural network system and allows the early stopping method to work.

### Experimental Examples

Hereinafter, experimental examples are provided to help understand the present invention. The following experimental examples are presented to help understand the invention, and the scope of the present invention is not limited to the experimental examples.

As anisotropic materials for learning, materials exhibiting plastic anisotropy, specifically two types of ultra-high strength steels (DP780 and TBF1050) and two types of aluminum alloys (AA7075 and AA 2090) were selected.

To verify the finite element simulation for the indentation as a means of database creation and to confirm the validity of the deep learning prediction results, a tensile test was performed on the anisotropic material for learning.

The anisotropic materials for learning were subjected to a tensile test to obtain tensile properties. The tensile properties included the tensile stresses (σ₀˜σ₉₀, σ_{b}) and r-values (r₀˜r₉₀, r_{b}) of the anisotropic materials for learning. Tensile tests of the anisotropic material for learning were performed as follows. First, each of the anisotropic materials for learning was manufactured into a tensile specimen with a thickness of 1 mm. The tensile specimen was subjected to a uniaxial tensile test using a universal tensile tester (Instron 5584, USA) according to the ASTM-E08 standard. The tensile specimen was subjected to quasi-static tensile tests in a rolling direction (RD), diagonal direction (DD), and transverse direction (TD) at a nominal strain rate of 10⁻³ s⁻¹.

The tensile test results were analyzed using an analysis solution, and a total of 19 parameters were obtained by combining 16 anisotropic parameters of the Poly6 model and 3 isotropic parameters of the Swift hardening model.

The indentation tests of the anisotropic materials for learning were performed as follows. Each of the anisotropic materials for learning was fabricated into a rectangular indentation specimen with a size of 60×50×1 mm. Before performing the indentation test, the surface of the indentation specimen was subjected to mechanical grinding and polishing using an alumina suspension containing alumina particles with a diameter of up to 1 μm. An instrumented indentation test was performed on the indentation specimen using an AIS3000 system (Frontics, Inc., Korea) with a load resolution of 0.05 N and a displacement resolution of 0.1 μm. In the indentation test, a spherical indenter of 250 μm radius was used. sufficient area below the indentation should be allowed so as to accurately describe the tensile behavior of bulk materials, so the indentation depth was controlled to a maximum of 150 μm using displacement control. To ensure reliability, the indentation was performed five times and the average value was obtained.

In addition, the tensile test and indentation test of the to-be-predicted anisotropic material were performed in the same manner.

To obtain the in-plane displacement field information and vertical displacement field information of the indentation specimen, images of the indentation specimen before and after the indentation test were obtained.

Obtaining the in-plane displacement field information of the indentation specimen requires a non-contact, non-destructive, and simple optical setup, so the Digital Image Correlation (DIC) technique used for strain rate measurement was used. A spot pattern was formed by spray painting the surface of the indentation specimen before indentation. The in-plane displacement field of the indentation specimen was calculated by obtaining digital images before and after deformation, and tracking each point of the images using the MATLAB DIC application.

The vertical displacement field was measured using a 3D laser scanning confocal microscope (LSCM) system (VK-X21 series, Keyence, Japan) equipped with a 10× objective lens. The 3D topography of the indentation may be reconstructed by obtaining multiple 2D images at regular intervals and accumulating them. Image processing was performed using VK-analyzer software, and pile-up/sink-in information was obtained by observing the vertical cross-section of a 3D terrain.

A finite element simulation for the anisotropic material for learning was performed using commercial ABAQUS software. A ¼ finite element indentation model consisting of 3D 8-node continuous brick elements (C3D8R) with reduced integration points was used. In the finite element simulation, the shape of the indenter was selected as a spherical indenter which is the same as the one used in the experiment. The Young's modulus of the spherical indenter was 700 GPa, and the Poisson's ratio thereof was 0.3. The specimen model for the finite element simulation had a radius and thickness of 3 mm, which can exclude geometrical effects compared to the indenter size and indentation depth. The mechanical boundary conditions of lateral axisymmetric and bottom fixation were imposed on the specimen model. As in the experiment, a displacement condition of 0.3 mm/min in the z-axis direction was applied to the indenter until the indentation depth reached a maximum of 150 μm. During indentation, the interaction between the indenter and the specimen surface was controlled using the surface-to-surface contact pair algorithm, normal hard contact, and tangential friction. Since the friction coefficient has a great influence on the load-depth curve in deep spherical indentations of 50 μm or more, 0.12 was used as the optimal friction coefficient. A preliminary investigation was conducted on contact area mesh improvement and convergence.

Table 1 shows the mechanical parameters of anisotropic materials for learning in the deep learning-based method of predicting the plastic properties of an anisotropic material by using indentation response data according to an embodiment of the present invention.

TABLE 1

| | a₁ | a₂ | a₃ | a₄ | a₅ | a₆ | a₇ | a₈ | | | |
|---|---|---|---|---|---|---|---|---|---|---|---|
| Material | a₉ | a₁₀ | a₁₁ | a₁₂ | a₁₃ | a₁₄ | a₁₅ | a₁₆ | k | e₀ | n |
| TBF1050 | 1 | −2.92 | 6.42 | −7.69 | 5.99 | −2.35 | 0.746 | 5.44 | 1717 | 0.006 | 0.16 |
| | −13.67 | 23.18 | −17.80 | 8.67 | 24.99 | −24.32 | 20.94 | 21.97 | | | |
| DP780 | 1 | −2.64 | 5.85 | −7.38 | 5.89 | −2.80 | 1.04 | 9.68 | 1315 | 0.005 | 0.14 |
| | −19.7 | 30.42 | −18.40 | 9.68 | 28.63 | −22.90 | 27.24 | 33.16 | | | |
| AA2090-T3 | 1 | −1.05 | 2.19 | −4.75 | 5.97 | −4.32 | 1.76 | 15.36 | 646 | 0.025 | 0.227 |
| | −3.63 | −10.88 | 5.15 | 16.06 | 7.43 | −9.04 | 85.58 | 117.42 | | | |
| AA7075-T6 | 1 | −2.41 | 7.28 | −11.18 | 8.38 | −3.18 | 1 | 8.97 | 882 | 0.045 | 0.154 |
| | −20.97 | 30.58 | −15.40 | 6.04 | 17.17 | −13.29 | 23.01 | 27 | | | |

Referring to Table 1, since a1=1 for all the anisotropic materials for learning, there are 18 independent parameters. Since these parameters are predicted based on the indentation response data, it is necessary to deeply consider the deformation field formed by the indentation. The parameter review was performed using a random generation method for each parameter, and a total of 50,000 data sets were generated.

Table 2 shows parameters input to the finite element simulation and ranges thereof, in the deep learning-based method of predicting the plastic properties of an anisotropic material by using indentation response data according to an embodiment of the present invention according to an embodiment of the present invention.

TABLE 2

| | | Model | | | | | |
|---|---|---|---|---|---|---|---|
| | | Swift hardening | | | Yield stress | | r-value |
| | | Parameter | | | | | |
| | | k | | | | σ₁₅~σ₉₀, | r₀~r₉₀, |
| | | (MPa) | ε0 | n | σ₀ | σ_{b} | r_{b} |
| Range | Minimum | 300 | 0.0001 | 0.01 | 1 | 0.8 | 0.2 |
| | Maximum | 2000 | 0.05 | 0.5 | | 1.2 | 2.0 |

In Table 2, the Young's modulus of the aluminum alloy was set to 70 GPa, and the Young's modulus of the iron alloy was set to 200 GPa. The Poisson's ratio of the aluminum alloy and iron alloy was set to 0.3.

Next, the finite element simulation was performed using the poly6 anisotropy parameter, elastic modulus, and isotropic hardening parameter (Swift hardening parameter) obtained from the tensile properties of the anisotropic material for learning.

The finite element simulation may be repeatedly performed to output output values. The output values included the load-depth curve, the in-plane displacement field and the vertical displacement field.

To perform subsequent deep learning, indentation response data for learning was obtained as the feature data through a process of extracting the feature data from the output values. The indentation response data for learning included indentation load data, radial displacement data, and vertical displacement data of the anisotropic material for learning.

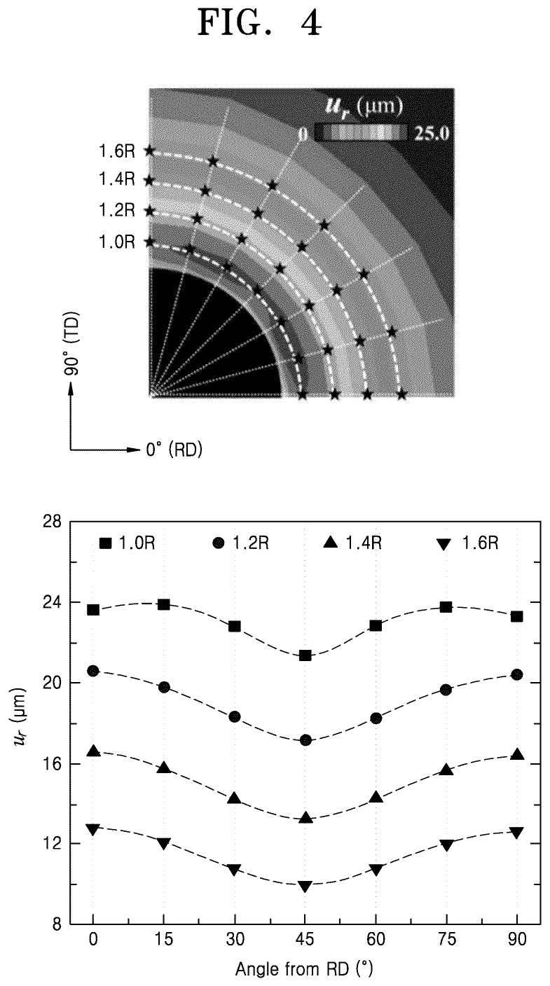

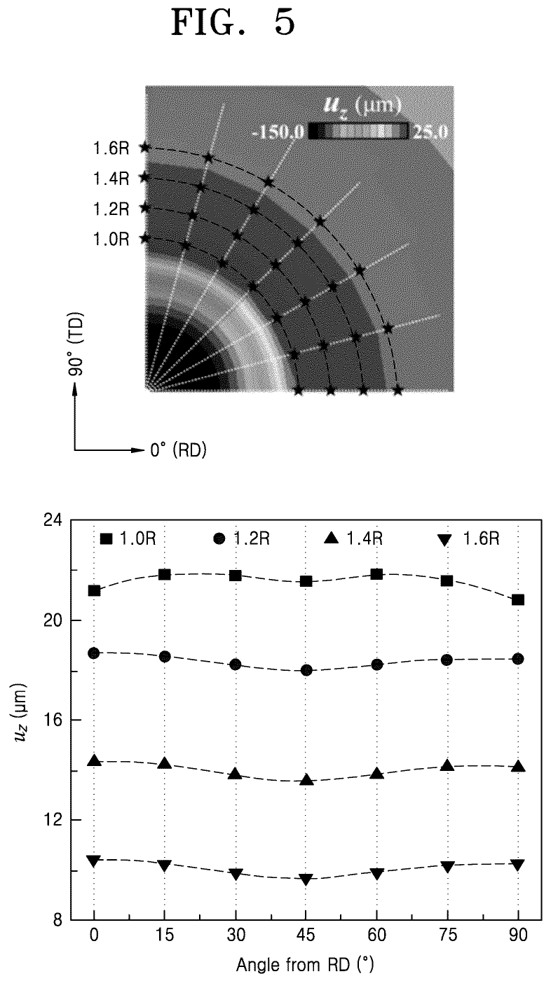

FIGS. 4 and 5 are graphs showing displacement due to the indentation calculated using the finite element simulation, in the deep learning-based method of predicting the plastic properties of an anisotropic material by using indentation response data according to an embodiment of the present invention.

FIGS. 4 and 5 respectively show the radial displacement (uᵣ) and vertical displacement (u_{z}) obtained by performing the finite element simulation for DP780 steel. Each of the radial displacement (uᵣ) and the vertical displacement (u_{z}) was obtained at various angles, for example, 0 degrees, 15 degrees, 30 degrees, 45 degrees, 60 degrees, 75 degrees, and 90 degrees, in the rolling direction (RD) from the residual strain field around the indentation. Each of the obtainment locations of the radial displacement and the vertical displacement is shown on an upper side. The obtainment location was set by changing the angle at 15-degree intervals from the rolling direction (RD) at a separation distance of multiples of the radius (R) of the indenter based on the indentation center. The separation distances of multiples were 1.0R, 1.2R, 1.4R, and 1.6R.

The radial displacement and the vertical displacement are the same value at the same separation distance regardless of the angle in the case of isotropic materials. However, it can be seen that different values are shown in anisotropic materials having plastic anisotropy, such as the DP780 steel. That is, even though it was obtained at the same distance from the indentation center, the deviation with different values appeared as the angle changed, and the minimum value was shown at 45 degrees from the rolling direction at all distances. The deviation was greater in the radial displacement. The behavior of the radial displacement and the behavior of the vertical displacement were similar at 1.2R, 1.4R, and 1.6R, while they were different at a distance of 1.0R. This anisotropic behavior was also similarly observed in TBF1050 steel, AA7075 aluminum alloy, and AA2090 aluminum alloy.

The plastic region of the uniaxial tensile curve in the rolling direction (RD) may be used as a reference state for determining the hardening parameters for each material. Conventionally, to define the anisotropic parameter of an anisotropic material, a yield stress and r-value obtained by performing a uniaxial tensile test at various angles were converted. However, According to the technical idea of the present invention, anisotropic parameters may be defined from the indentation test results as shown in FIGS. 4 and 5.

This similar behavior means that the data shown in FIGS. 4 and 5 are not all valid as input values that are input to deep learning. Therefore, to verify the valid data, the linearity of the radial displacement and the vertical displacement is examined.

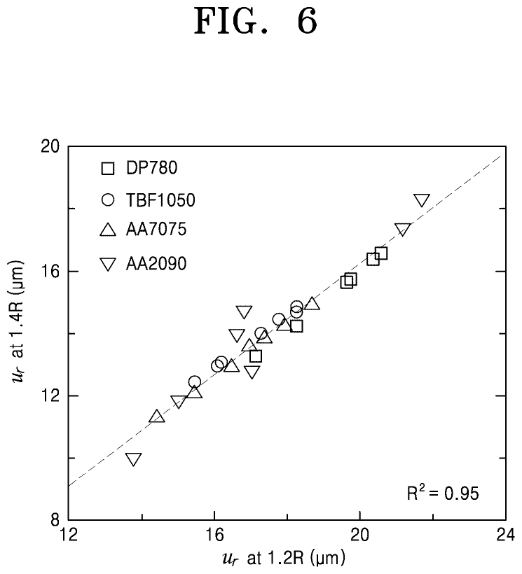

FIGS. 6 to 8 illustrate the linearity between displacements due to indentation obtained using a finite element simulation, in the deep learning-based method of predicting the plastic properties of an anisotropic material by using indentation response data according to an embodiment of the present invention.

FIG. 6 shows a correlation between the radial displacement (uᵣ) of the 1.2R distance and the radial displacement of the 1.4R distance which are obtained for the DP780 steel, the TBF1050 steel, the AA7075 aluminum alloy, and the AA2090 aluminum alloy. The correlation between the radial displacements shows strong linearity at the level of R²=95%.

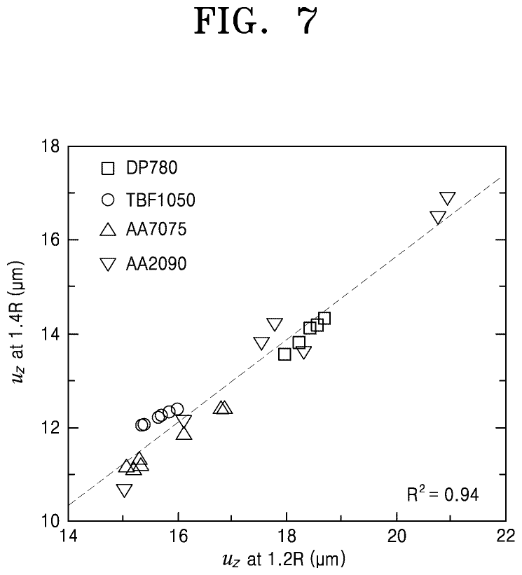

FIG. 7 shows a correlation between the vertical displacement (u_{z}) of the 1.2R distance and the vertical displacement of the 1.4R distance which are obtained for the DP780 steel, the TBF1050 steel, the AA7075 aluminum alloy, and the AA2090 aluminum alloy. The correlation between the vertical displacements shows strong linearity at the level of R²=94%.

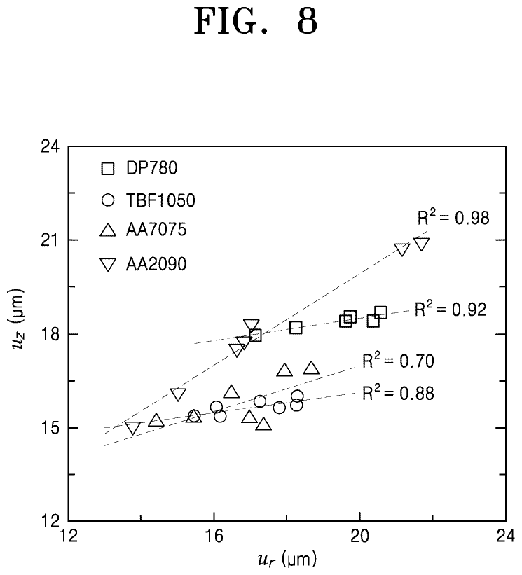

FIG. 8 shows a correlation between the radial displacement (uᵣ) of the 1.2R distance and the vertical displacement (u_{z}) of the 1.2R distance which are obtained for the DP780 steel, the TBF1050 steel, the AA7075 aluminum alloy, and the AA2090 aluminum alloy. The DP780 steel shows linearity at the level of R²=92%, the TBF1050 steel shows linearity at the level of R²=88%, the AA7075 aluminum alloy shows linearity at the level of R²=70%, and the AA2090 aluminum alloy shows linearity at the level of R²=98%. When the results of the four materials are integrated and calculated, linearity at the level of the average R²=87% is shown. When comparing the results of FIGS. 6 and 7, the linearity is somewhat low, but strong linearity is still shown.

Referring to the results of FIGS. 6 to 8, for example, 1.2R radial displacement, 1.4R radial displacement, 1.6R radial displacement, 1.2R vertical displacement, 1.4R vertical displacement, and 1.6R vertical displacement all have linearity, so one of the data sets may be selected as an input value of a representative data set. However, due to the pile-up and sink-in phenomena formed around the indentation, the radial displacement measured at 1.0R may contain a large error. Therefore, the 1.0R vertical displacement, which exhibits different behavior, may be selected as an additional input value. For example, the 1.4R radial displacement and the 1.0R vertical displacement may be selected as input values.

Hereinafter, a finite element simulation for the load on indentation depth derived from the load-depth curve, the radial displacement according to the angle calculated from the in-plane strain field, and the vertical displacement according to the angle calculated from the vertical strain field will be verified by comparing experimental results.

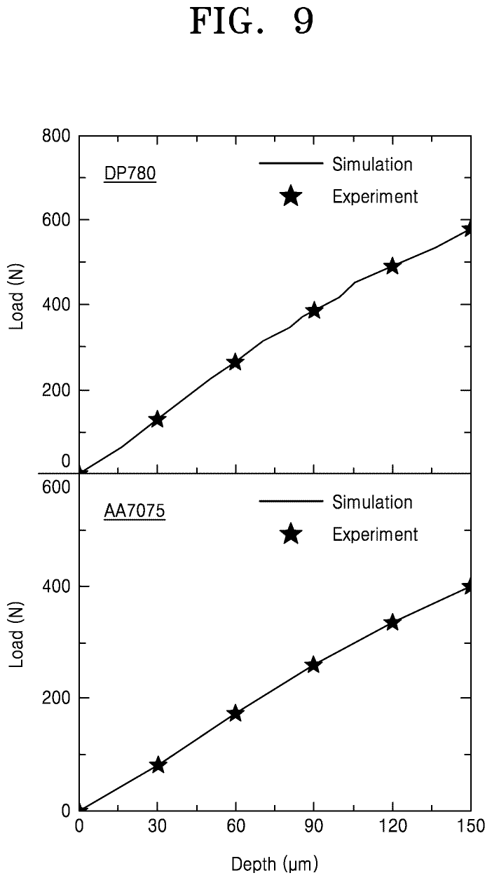

FIGS. 9 to 11 illustrate graphs comparing finite element simulation results and actual simulation results, in the deep learning-based method of predicting the plastic properties of an anisotropic material by using indentation response data according to an embodiment of the present invention.

FIG. 9 shows the load for the indentation depth respectively obtained from the finite element simulation results and experimental results for the DP780 steel and the AA7075 aluminum alloy. The indentation depth was changed to 30 μm, 60 μm, 90 μm, 120 μm, and 150 μm in 30 μm increments, and the indentation load at each indentation depth was measured. It was confirmed that the finite element simulation results and experimental results for the load-depth behavior of the DP780 steel and the AA7075 aluminum alloy were very consistent. Therefore, since the validity of the indentation model can be guaranteed, the load-depth data derived from the finite element simulation can be used as input values in deep learning.

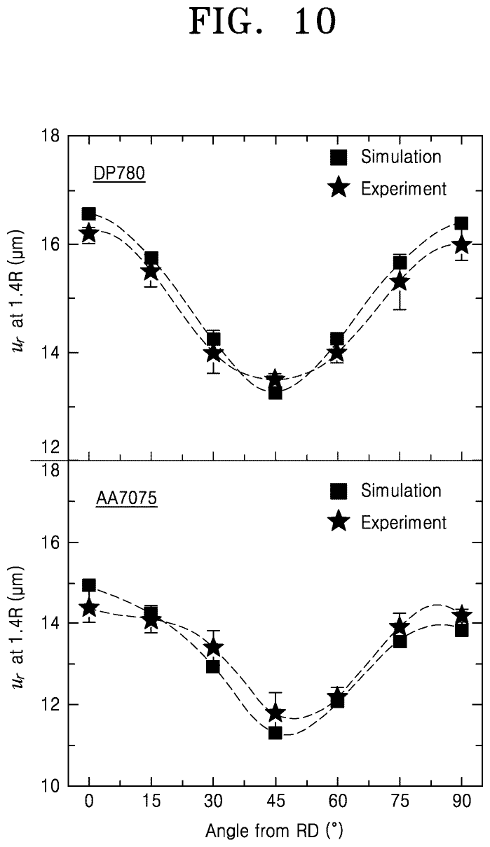

FIG. 10 shows the radial displacement (uᵣ) at a distance of 1.4R for an angle from a reference direction, for example, from the rolling direction (RD), obtained from each of the finite element simulation results and experimental results for the DP780 steel and the AA7075 aluminum alloy. The experimental results of the radial displacement were measured using the Digital Image Correlation (DIC) method. The dotted line was obtained using the cubic spline interpolation method. Due to the anisotropy of the DP780 steel and the AA7075 aluminum alloy, the radial displacement varied with angle and had the lowest radial displacement at 45 degrees, which was the same in both the finite element simulation results and the experimental results. It was confirmed that the finite element simulation results and experimental results for the radial displacement behavior for the angle for the DP780 steel and the AA7075 aluminum alloy were in good overall agreement. Therefore, since the validity of the indentation model can be guaranteed, the radial displacement data for the angle acquired by the finite element simulation can be used as an input value in deep learning.

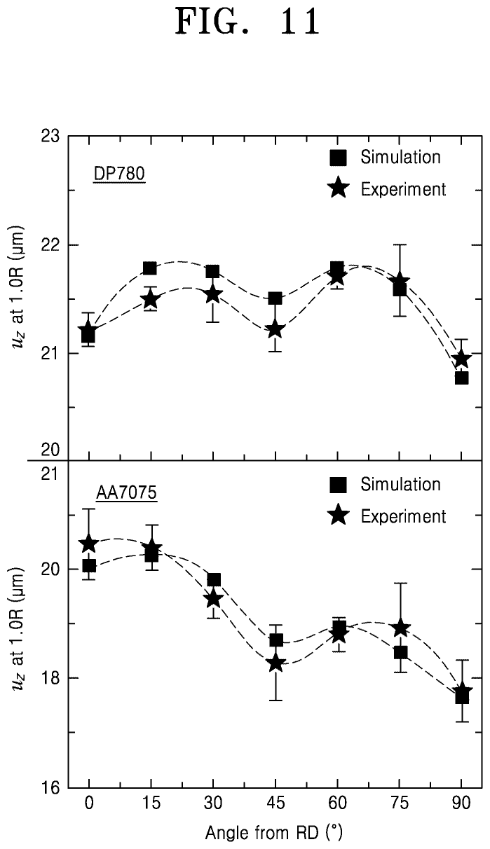

FIG. 11 shows the vertical displacement (u_{z}) at a distance of 1.0R from a reference direction, for example, the rolling direction (RD), obtained from each of the finite element simulation results and the experimental results for the DP780 steel and the AA7075 aluminum alloy. The experimental results of vertical displacement were obtained using a confocal laser scanning microscope (CLSM). The dotted line was obtained using the cubic spline interpolation method. Due to the anisotropy of the DP780 steel and the AA7075 aluminum alloy, the vertical displacement varied with angle and had the lowest vertical displacement at 90 degrees, which was the same in both the finite element simulation results and the experimental results. It was confirmed that the finite element simulation results and experimental results for the vertical displacement behavior for the angle for the DP780 steel and the AA7075 aluminum alloy were in good overall agreement. Therefore, the validity of the indentation model can be guaranteed, so that the vertical displacement data for the angle acquired by the finite element simulation can be used as an input value in deep learning.

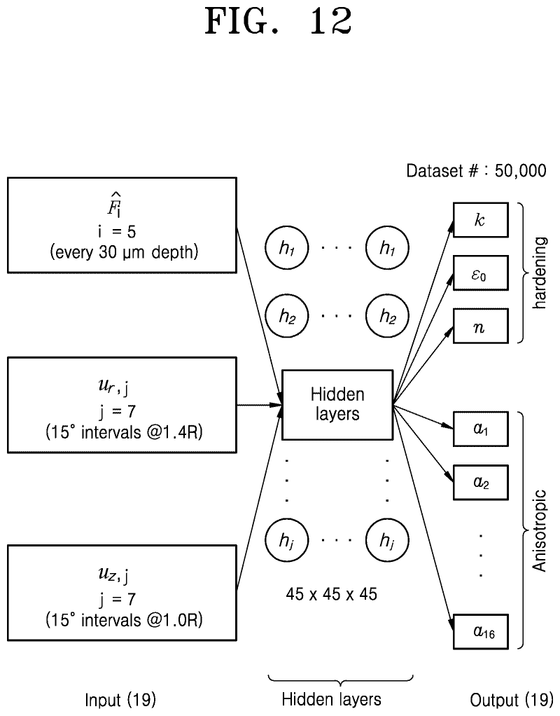

FIG. 12 illustrates a schematic diagram of an artificial neural network that performs deep learning, in the deep learning-based method of predicting the plastic properties of an anisotropic material by using indentation response data according to an embodiment of the present invention.

Referring to FIG. 12, the input values in the artificial neural network may be composed of indentation response data, and may be composed of, for example, a total of 19. The input values may be composed of a total of 5 loads (Fᵢ, i=5) extracted from the load-depth curve at indentation depths at intervals of 30 μm, a total of 7 radial displacements (u_{r, j}, j=7) at intervals of 15 degrees from 0 to 90 degrees from the rolling direction at a distance of 1.4R from the indentation center, and a total of 7 vertical displacements (u_{z, j}, j=7) at intervals of 15 degrees from 0 to 90 degrees from the rolling direction at a distance of 1.0R from the indentation center.

In the artificial neural network, the output values may be composed of plastic properties data, and for example, may be composed of a total of 19. The output values may be composed of 3 hardening parameters (k, ε₀, n) and 16 poly6 anisotropy parameters (a₁˜a₁₆).

In the artificial neural network, the hidden layer may be configured as a 3-layer model with 45 neurons per layer derived as a result of hyperparameter optimization.

When the artificial neural network performs the prediction step after deep learning, the indentation response data of the to-be-predicted anisotropic material may be set as input values, and the predicted plastic properties of the to-be-predicted anisotropic material data may be set as output values.

The numbers and configurations of the input values, the output values, and the hidden layers are exemplary, and the technical idea of the present invention is not limited thereto.

The above artificial neural network may apply the feed-forward & back propagation method and may be configured with the Levenberg-Marquardt (LM) algorithm.

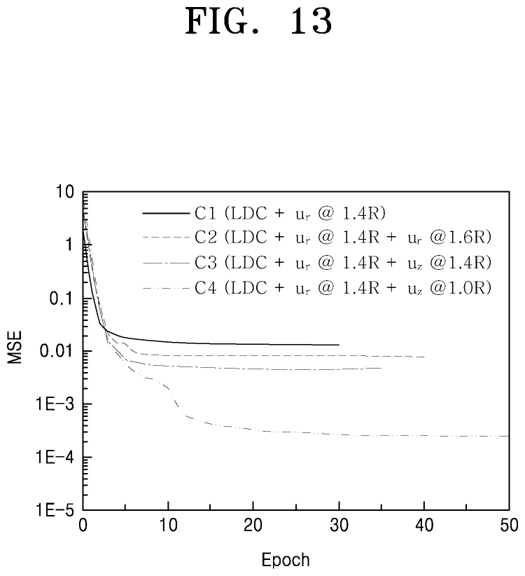

FIG. 13 illustrates the effect of data linearity on the performance of an artificial neural network performing deep learning, in the deep learning-based method of predicting the plastic properties of an anisotropic material by using indentation response data according to an embodiment of the present invention.

FIG. 13 shows the mean square errors (MSE) by epoch for data sets C1 to C4 which were input as input values to the artificial neural network. The data set C1 includes only 1.4R radial displacement (uᵣ) and do not include vertical displacement (u_{z}), the data set C2 includes 1.4R radial displacement and 1.6R radial displacement, the data set C3 includes 1.4R radial displacement and 1.4R vertical displacement, and the data set C4 includes 1.4R radial displacement and 1.0R vertical displacement.

Depending on the data sets, the mean square error reduction trend in the deep learning process of the artificial neural network was different. In the case of the data set C1, since the vertical displacement is not included, the amount of data for deep learning is relatively insufficient, resulting in a high mean square error. In addition, as described above, the data corresponding to 1.2R, 1.4R, and 1.6R have linearity, and the data corresponding to 1.0R do not have linearity for data corresponding to other distances, so it can be seen that the C4 including 1.4R radial displacement and 1.0R a vertical displacement excluding linearity has a low mean square error.

Therefore, it is analyzed that the performance of the artificial neural network increases as the data linearity is absent or low. In the case of configuring a data set that excludes linearity as much as possible, such as the C4, the prediction of plastic anisotropy using an artificial neural network may be more precise. Therefore, it is analyzed that the selection of 1.4R radial displacement and 1.0R vertical displacement as input values is appropriate.

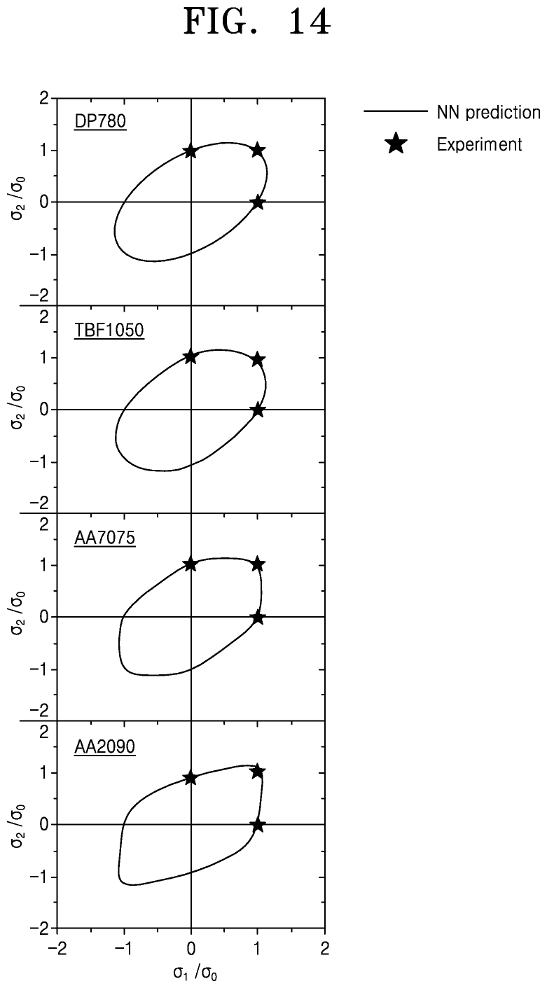

FIGS. 14 to 16 illustrate graphs comparing plasticity characteristic prediction results, obtained using an artificial neural network that is deep-learned by the deep learning-based method of predicting the plastic properties of an anisotropic material by using indentation response data according to an embodiment of the present invention, and experimental results.

In FIGS. 14 to 16, for the DP780 steel and the AA7075 aluminum alloy, the indentation data measured in the actual indentation test were used as input values for the deep learning of the artificial neural network, and for the TBF1050 steel and the AA2090 aluminum alloy, the indentation data derived using a finite element simulation were used as input values for the deep learning of the artificial neural network.

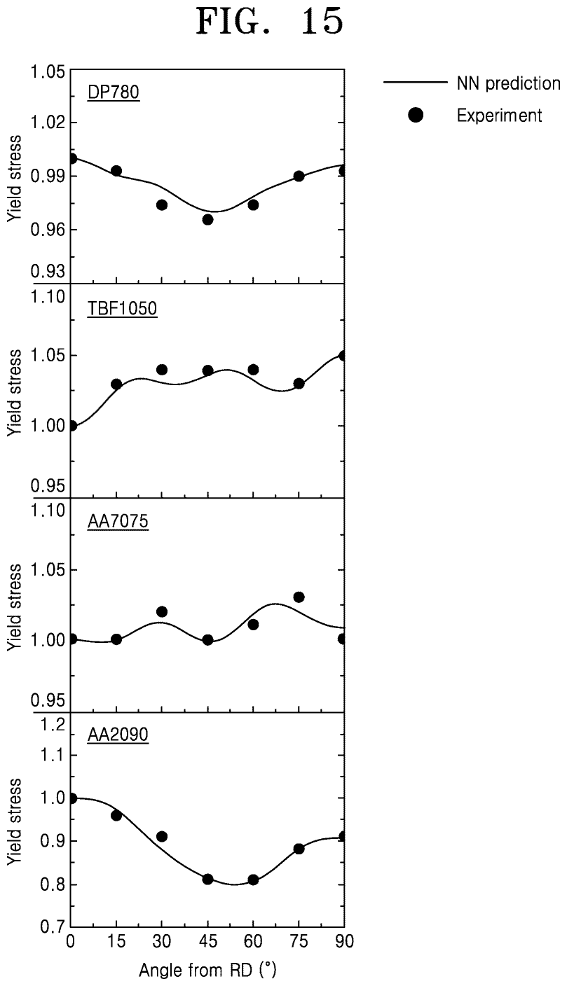

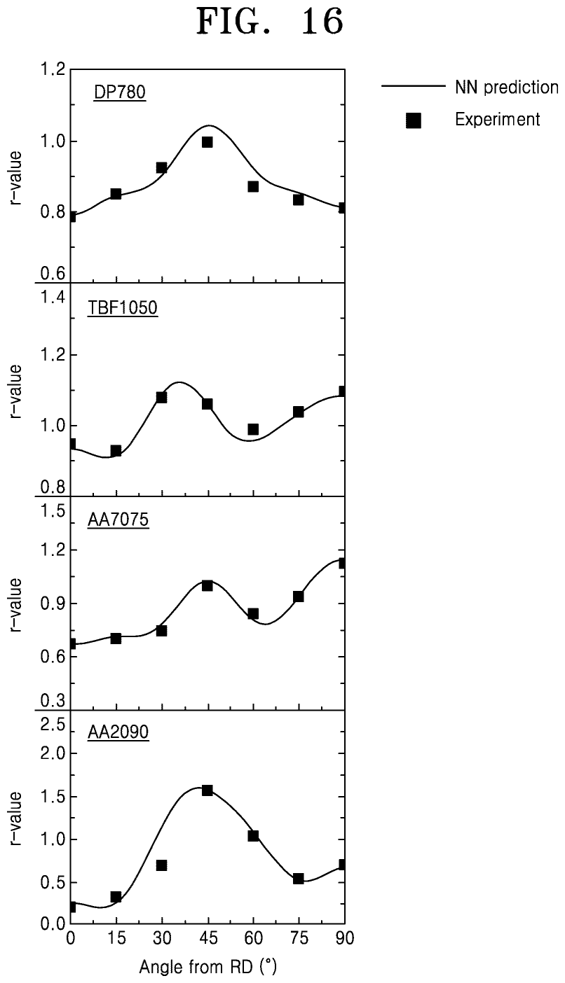

FIGS. 14 to 16 illustrate results of plastic properties predicted from the learned artificial neural network. Specifically, FIG. 14 shows the yield locus derived from the correlation of σ2/σ0 to σ1/σ0. FIG. 15 shows the yield stress for an angle from the reference direction. FIG. 16 shows the r-value for an angle from the reference direction. When the artificial neural network is deep-learned using the indentation data obtained by the finite element simulation or when the artificial neural network is deep-learned using the indentation data obtained by the actual indentation experiment, it can be seen that the plastic properties, such as the yield trajectory, the yield stress, and the r-value, predicted by the deep-learned artificial neural network are very consistent with the actual simulation results.

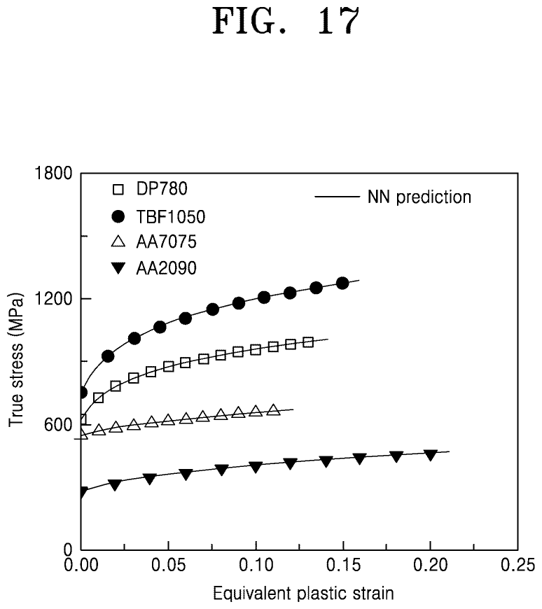

FIG. 17 illustrates plastic properties predicted using an artificial neural network deep-learned by the deep learning-based method of predicting the plastic properties of an anisotropic material by using indentation response data according to an embodiment of the present invention.

FIG. 17 shows true stress for equivalent plastic strain predicted using the deep-learned artificial neural network for the DP780 steel, the TBF1050 steel, the AA7075 aluminum alloy, and the AA2090 aluminum alloy. This corresponds to the Swift hardening relation of Mathematical Equation 2. The data points are based on actual simulation results, and the solid line is a predicted curve (NN prediction) predicted by the deep-learned artificial neural network. In all cases, it can be seen that the experimental results and the predicted curves are in good agreement.

Accordingly, by deep learning the artificial neural network with radial displacement and vertical displacement at various indentation distances along with the load-distance curve as input values, many parameters explaining plastic anisotropy can be successfully determined. Therefore, metal forming simulations for various materials can be implemented reliably and efficiently by using the deep learning-based method of predicting the plastic properties of a metal material according to the present invention.

It will be obvious to those skilled in the art, to which the technical idea of the invention pertains, that the technical idea of the invention described above is not limited to the above-described embodiments and the accompanying drawings and various substitutions, modifications, and changes are possible within the scope of the technical idea of the invention.

### INDUSTRIAL APPLICABILITY

Metal forming simulations for various materials can be implemented reliably and efficiently by using a deep learning-based method of predicting the plastic properties of a metal material according to the present invention.

1. A deep learning-based method of predicting the plastic properties of an anisotropic material by using indentation response data, the deep learning-based method comprising:
preparing a plurality of data sets, which are composed of indentation response data for learning and plastic properties data for learning, about an anisotropic material for learning;
performing deep learning on a computer system by using the indentation response data for learning as input values and using the plastic properties data for learning as output values;
providing actual indentation response data about a to-be-predicted anisotropic material; and
predicting plastic properties of the to-be-predicted anisotropic material by inputting the actual indentation response data into the deep-learned computer system.

2. The deep learning-based method according to claim 1, wherein the preparing comprising:
providing tensile properties of the anisotropic material for learning;
obtaining a poly6 anisotropy parameter, elastic modulus, and isotropic hardening parameter from the tensile properties of the anisotropic material for learning;
performing a finite element simulation using the poly6 anisotropy parameter, the elastic modulus, and the isotropic hardening parameter; and
obtaining the indentation response data for learning about the anisotropic material for learning as a result of performing the finite element simulation.

3. The deep learning-based method according to claim 2, wherein the tensile properties comprise a tensile stress and r-value of the anisotropic material for learning.

4. The deep learning-based method according to claim 2, wherein the poly6 anisotropy parameter is obtained from the following equation:
$$f = {a}_{1} {\sum }_{xx}^{6} + {α}_{2} {\sum }_{xx}^{5} {\sum }_{yy} + {α}_{3} {\sum }_{xx}^{4} {\sum }_{yy}^{2} + {α}_{4} {\sum }_{xx}^{3} {\sum }_{yy}^{3} + {α}_{5} {\sum }_{xx}^{2} {\sum }_{yy}^{4} + {α}_{6} {\sum }_{xx} {\sum }_{yy}^{5} + {α}_{7} {\sum }_{yy}^{6} + ( {α}_{8} {\sum }_{xx}^{4} + {α}_{9} {\sum }_{xx}^{3} {\sum }_{yy} + {α}_{10} {\sum }_{xx}^{2} {\sum }_{yy}^{2} + {α}_{11} {\sum }_{xx} {\sum }_{yy}^{3} + {α}_{12} {\sum }_{yy}^{2} ) {\sum }_{xy}^{2} + ( {α}_{13} {\sum }_{xx}^{2} + {α}_{14} {\sum }_{xx} {\sum }_{yy} + {α}_{15} {\sum }_{yy}^{2} ) {( {\sum }_{xy}^{2} )}^{2} + {{α}_{16} ( {σ}_{xy}^{2} + {σ}_{yz}^{2} + {σ}_{zx}^{2} )}^{3} = {σ}_{0}^{6}$$
$$\{ \begin{matrix}{\sum }_{xx} = {σ}_{xx} - {σ}_{zz} \\ {\sum }_{yy} = {σ}_{yy} - {σ}_{zz} \\ {\sum }_{xy}^{2} = {σ}_{xy}^{2} + {σ}_{yz}^{2} + {σ}_{zx}^{2}\end{matrix}$$
where σₓₓ, σ_{yy} and σ_{zz} are vertical stresses related to the vertical direction, and σ_{xy}, σ_{yz} and σ_{zx} are shear stresses.

5. The deep learning-based method according to claim 2, wherein the elastic modulus comprises Young's modulus (E) and Poisson's ratio (v) of the anisotropic material for learning.

6. The deep learning-based method according to claim 1, wherein the isotropic hardening parameter comprises a strength coefficient (k), strain parameter (ε₀), and strain hardening exponent (n) obtained from the following equation:
$${\overline{σ}}_{Swift} ( \overline{{ε}_{p}} ) = {k ( {ε}_{0} + \overline{{ε}_{p}} )}^{n}$$
where σ̅_{Swift} is a Swift effective stress, and ε̅ₚ is an equivalent plastic strain.

7. The deep learning-based method according to claim 2, wherein, after the obtaining of the poly6 anisotropy parameter, evaluating whether the poly6 anisotropy parameter of the anisotropic material for learning satisfies a convexity for a yield criterion of the anisotropic material for learning is further comprised.

8. The deep learning-based method according to claim 2, wherein output values obtained by performing the finite element simulation comprise a load-depth curve, in-plane displacement field information, and vertical displacement field information from results of the anisotropic material for learning.

9. The deep learning-based method according to claim 1, wherein the indentation response data for learning comprises indentation load data, radial displacement data, and vertical displacement data for an indentation formed by indenting the anisotropic material for learning.

10. The deep learning-based method according to claim 9, wherein the indentation load data comprises a load value for the depth of the indentation.

11. The deep learning-based method according to claim 9, wherein the radial displacement data comprises a radial displacement value for an angle from a reference direction of the indentation.

12. The deep learning-based method according to claim 11, wherein the radial displacement data comprises a radial displacement value at a separation distance that is a multiple of a radius (R) of the indenter from a center of the indentation.

13. The deep learning-based method according to claim 9, wherein the vertical displacement data comprises a vertical displacement value for the angle from the reference direction of the indentation.

14. The deep learning-based method according to claim 13, wherein the vertical displacement data comprises a vertical displacement value at a separation distance that is a multiple of a radius (R) of the indenter from a center of the indentation.

15. The deep learning-based method according to claim 1, wherein, after the predicting, comparing predicted plastic properties of the to-be-predicted anisotropic material with actual plastic properties of the to-be-predicted anisotropic material is further comprised.
